(19) United States
(12) Patent Application Publication
(10) Pub. No.: US 2026/0311809 A1
(43) Pub. Date: Oct. 8, 2026

(54) BLIND DENOISING OF SEQUENCING DATA

(71) Applicant: Genentech, Inc., South San Francisco, CA (US)

(72) Inventors: Ryan Lewis KELLY, San Mateo, CA (US); Jae Hyeon LEE, Boston, MA (US); Nathan Hoyen NG, Cambridge, MA (US); Ji Won PARK, Foster City, CA (US); Kyunghyun CHO, New York, NY (US)

(21) Appl. No.: 19/093,056

(22) Filed: Mar. 27, 2025

Related U.S. Application Data
(63) Continuation of application No. PCT/US2023/075233, filed on Sep. 27, 2023.
(60) Provisional application No. 63/377,337, filed on Sep. 27, 2022.
(60) Provisional application No. 63/378,460, filed on Oct. 5, 2022.

(51) Int. Cl. G16B 30/10; G16B 40/00

(52) U.S. Cl. CPC G16B 30/10; G16B 40/00

(57) ABSTRACT

A method for blind denoising of sequencing data may include receiving a plurality of read sequences associated with a scaffold sequence. Each read sequence may be a repeat of the scaffold sequence, at least one of which is a noisy read sequence that fails to match the scaffold sequence. A sequence denoising model may be applied to encode the plurality of read sequences, and generate, based on an encoding of the plurality of read sequences, a denoised scaffold sequence that corresponds to the scaffold sequence. The denoised scaffold sequence may be generated absent the scaffold sequence. A molecule associated with the scaffold sequence may be analyzed based on the denoised scaffold sequence. Related systems and computer program products are also provided.

### CROSS REFERENCE TO RELATED APPLICATIONS

This application claims priority to U.S. Provisional Application No. 63/377,337, entitled “BLIND DENOISING OF SEQUENCING DATA” and filed on Sep. 27, 2022, and U.S. Provisional Application No. 63/378,460, entitled “BLIND DENOISING OF SEQUENCING DATA” and filed on Oct. 5, 2022, the disclosures of which are incorporated herein by reference in their entirety.

### TECHNICAL FIELD

The subject matter described herein relates generally to the gene sequencing and more specifically to techniques for denoising sequencing data.

### INTRODUCTION

In the context of genetics and biochemistry, the term “sequencing” refers to a variety of techniques for determining the primary structure of an unbranched biopolymer. For example, sequencing a nucleic acid molecule (e.g., deoxyribonucleic acid (DNA), ribonucleic acid (RNA), and/or the like) or variants or derivatives thereof (e.g., single stranded DNA) may include determining the sequence of nucleotide bases forming the nucleic acid molecule. In the case of DNA, the sequencing of a DNA fragment may include determining the order of guanine (G), adenine (A), cytosine (C), and thymine (T) in the DNA fragment. Meanwhile, the sequencing of an RNA fragment may include determining the order of guanine (G), adenine (A), cytosine (C), and uracil (U) in the RNA fragment.

### SUMMARY

Systems, methods, and articles of manufacture, including computer program products, are provided for blind denoising of sequencing data including multiple read sequences associated with a scaffold sequence absent the scaffold sequence. In one aspect, there is provided a system for blinding denoising of sequencing data. The system may include at least one processor and at least one memory. The at least one memory may include program code that provides operations when executed by the at least one processor. The operations may include: receiving a plurality of read sequences associated with a scaffold sequence, each read sequence of the plurality of read sequences comprising a repeat of the scaffold sequence, and at least one read sequence of the plurality of read sequences being a noisy read sequence that fails to match the scaffold sequence; applying a sequence denoising model trained to determine an encoding associated with the plurality of read sequences, and generate, based at least on the encoding associated with the plurality of read sequences, a denoised scaffold sequence corresponding to the scaffold sequence; and analyzing, based at least on the denoised scaffold sequence, a molecule associated with the scaffold sequence.

In another aspect, there is provided a method for blind denoising of sequencing data. The method may include: receiving a plurality of read sequences associated with a scaffold sequence, each read sequence of the plurality of read sequences comprising a repeat of the scaffold sequence, and at least one read sequence of the plurality of read sequences being a noisy read sequence that fails to match the scaffold sequence; applying a sequence denoising model trained to determine an encoding associated with the plurality of read sequences, and generate, based at least on the encoding associated with the plurality of read sequences, a denoised scaffold sequence corresponding to the scaffold sequence; and analyzing, based at least on the denoised scaffold sequence, a molecule associated with the scaffold sequence.

In another aspect, there is provided a computer program product for blind denoising of sequencing data. The computer program product may include a non-transitory computer readable medium storing instructions that cause operations when executed by at least one data processor. The operations may include: receiving a plurality of read sequences associated with a scaffold sequence, each read sequence of the plurality of read sequences comprising a repeat of the scaffold sequence, and at least one read sequence of the plurality of read sequences being a noisy read sequence that fails to match the scaffold sequence; applying a sequence denoising model trained to determine an encoding associated with the plurality of read sequences, and generate, based at least on the encoding associated with the plurality of read sequences, a denoised scaffold sequence corresponding to the scaffold sequence; and analyzing, based at least on the denoised scaffold sequence, a molecule associated with the scaffold sequence.

In some variations of the methods, systems, and non-transitory computer readable media, one or more of the following features can optionally be included in any feasible combination.

In some variations, the sequence denoising model may generate the denoised scaffold sequence absent the scaffold sequence.

In some variations, the sequence denoising model may generate the denoised scaffold sequence by at least encoding the plurality of read sequences to generate a plurality of embeddings, determining an aggregate embedding corresponding to an aggregate of the plurality of embeddings, and decoding the aggregate embedding to generate the denoised scaffold sequence.

In some variations, the sequence denoising model may include a sequence encoder trained to generate the plurality of embeddings by at least encoding the plurality of read sequences. The sequence encoder may be trained to increase, when encoding the plurality of read sequences, a similarity between the plurality of embeddings by at least reducing an edit distance between the plurality of embeddings when encoding the plurality of read sequences.

In some variations, the sequence encoder may be trained to generate, for each read sequence of the plurality of read sequences, a corresponding embedding in a latent space.

In some variations, the latent space may be a topological space occupied by a reduced dimension representation of the plurality of read sequences.

In some variations, the sequence encoder may be a transformer.

In some variations, the sequence denoising model may include a sequence decoder trained to generate the denoised scaffold sequence by least decoding the aggregate embedding to generate the denoised scaffold sequence.

In some variations, the sequence decoder may include a transformer.

In some variations, the sequence denoising model may include a set encoder trained to determine the aggregate embedding.

In some variations, the set encoder may be trained to reduce a first distance between the aggregate embedding and the plurality of embeddings in a latent space when determining the aggregate embedding. The set encoder may be further trained to reduce a second distance between the denoised scaffold sequence and the plurality of read sequences in a sequence space when determining the aggregate embedding.

In some variations, the set encoder may include a set transformer.

In some variations, the noisy read sequence may include at least one insertion, deletion, or substitution of a nucleobase type included in the scaffold sequence.

In some variations, an identity of the molecule associated with the scaffold sequence may be determined based at least on the denoised scaffold sequence.

In some variations, a binding specificity of an antibody may be determined based at least on the identity of the molecule conjugated to the antibody.

In some variations, a gene expressing a T cell receptor (TCR) may be identified based at least on the identity of the molecule conjugated to the T cell receptor (TCR).

In some variations, the scaffold sequence may be associated with a nucleic acid sequence label conjugated to (i) a heavy chain or a light chain of a B cell receptor or (ii) an alpha chain and/or a beta chain of a T-cell receptor.

In some variations, the scaffold sequence may be a deoxyribonucleic acid (DNA) sequence or a ribonucleic acid (RNA) sequence.

In some variations, the sequence denoising model may be trained, based at last on a training set, to generate one or more denoised scaffold sequences while one or more corresponding scaffold sequences are unknown. A performance of the sequence denoising model may be determined while the one or more corresponding scaffold sequences are unknown. Upon determining that the performance of the sequence denoising model satisfies one or more thresholds, the sequence denoising model may be applied to determine the encoding associated with the plurality of read sequences, and generate, based at least on the encoding associated with the plurality of read sequences, the denoised scaffold sequence corresponding to the scaffold sequence.

In some variations, a leave-one-out edit distance of the sequence denoising model may be determined by at least determining an edit distance between one read sequence from the training set and a denoised scaffold sequence generated by the sequence denoising model based on one or more remaining read sequences in the training set. The performance of the sequence denoising model may be determined based at least on the leave-one-out edit distance.

Implementations of the current subject matter can include, but are not limited to, methods consistent with the descriptions provided herein as well as articles that comprise a tangibly embodied machine-readable medium operable to cause one or more machines (e.g., computers, etc.) to result in operations implementing one or more of the described features. Similarly, computer systems are also described that may include one or more processors and one or more memories coupled to the one or more processors. A memory, which can include a non-transitory computer-readable or machine-readable storage medium, may include, encode, store, or the like one or more programs that cause one or more processors to perform one or more of the operations described herein. Computer implemented methods consistent with one or more implementations of the current subject matter can be implemented by one or more data processors residing in a single computing system or multiple computing systems. Such multiple computing systems can be connected and can exchange data and/or commands or other instructions or the like via one or more connections, including, for example, to a connection over a network (e.g. the Internet, a wireless wide area network, a local area network, a wide area network, a wired network, or the like), via a direct connection between one or more of the multiple computing systems, etc.

The details of one or more variations of the subject matter described herein are set forth in the accompanying drawings and the description below. Other features and advantages of the subject matter described herein will be apparent from the description and drawings, and from the claims. While certain features of the currently disclosed subject matter are described for illustrative purposes in relation to the denoising of sequencing data associated with deoxyribonucleic acid (DNA) and ribonucleic acid (RNA), it should be readily understood that such features are not intended to be limiting. The claims that follow this disclosure are intended to define the scope of the protected subject matter.

### DESCRIPTION OF DRAWINGS

The accompanying drawings, which are incorporated in and constitute a part of this specification, show certain aspects of the subject matter disclosed herein and, together with the description, help explain some of the principles associated with the disclosed implementations. In the drawings,

FIG. 1A depicts a system diagram illustrating an example of a sequencing system, in accordance with some example embodiments;

FIG. 1B depicts a flowchart illustrating an example of a process for denoising multiple read sequences associated with a scaffold sequence absent the scaffold sequence, in accordance with some example embodiments;

FIG. 2 depicts a schematic diagram illustrating an example of blind denoising in which multiple read sequences associated with a scaffold sequence are denoised absent the scaffold sequence, in accordance with some example embodiments;

FIG. 3A depicts a schematic diagram illustrating an example of a latent space, in accordance with some example embodiments;

FIG. 3B depicts a schematic diagram illustrating an example of a sequence denoising model generating a latent space representation of each noisy read sequence, in accordance with some example embodiments;

FIG. 3C depicts a schematic diagram illustrating an example of a sequence denoising model generating a set encoding of the latent space representations of multiple noisy read sequences, in accordance with some example embodiments;

FIG. 3D depicts a schematic diagram illustrating an example of latent space regularization, in accordance with some example embodiments;

FIG. 3E depicts a schematic diagram illustrating an example of leave-one-out (LOO) edit distance, in accordance with some example embodiments;

FIG. 4 depicts a schematic diagram illustrating a loss associated with an example of a sequence denoising model, in accordance with some example embodiments;

FIG. 5 depicts an example use case in which the read sequences associated with a light chain of an antibody are denoised using a sequence denoising model and a conventional multi-sequence alignment based denoising technique, in accordance with some example embodiments;

FIG. 6A depicts graphs illustrating a comparison of the respective performances of a sequence denoising model and a conventional multi-sequence alignment based denoising technique, in accordance with some example embodiments;

FIG. 6B depicts graphs illustrating another comparison of the respective performances of a sequence denoising model and a conventional multi-sequence alignment based denoising technique, in accordance with some example embodiments;

FIG. 6C depicts graphs illustrating another comparison of the respective performances of a sequence denoising model and a conventional multi-sequence alignment based denoising technique, in accordance with some example embodiments; and

FIG. 7 depicts a block diagram illustrating an example of a computing system, in accordance with some example embodiments.

When practical, similar reference numbers denote similar structures, features, or elements.

### DETAILED DESCRIPTION

The sequencing of nucleic acid molecules, such as deoxyribonucleic acid (DNA) and ribonucleic acid (RNA), plays a salient role in a variety of biotechnology pursuits. As one example use case in the context of large molecule drug discovery, the binding non-specificity (or poly-specificity) of antibodies, which is an undesirable biophysical property in which an antibody is capable of binding to the epitopes of many different antigens, may be evaluated by sequencing the nucleic acid sequence labels (e.g., plasmid, oligonucleotide, and/or the like) conjugated to each antibody expressed by yeast cells that have been treated to display individual antibodies from a library of different antibodies. For example, once the labeled antibodies have been exposed to one or more target and non-target antigens (e.g., poly-specific reagent), fluorescence activated cell sorting (FACS) may be performed to sort binding antibodies from non-binding antibodies before the nucleic acid sequence labels of the binding and non-binding antibodies are sequenced. Nevertheless, existing sequencing techniques for sequencing the nucleic acid sequence labels are associated with critical limitations. For instance, some next generation sequencing techniques are unable to resolve long sequences of nucleotide bases (e.g., sequences of more than 550 base pairs) whereas the heavy chain and light chain of an antibody includes approximately 700 base pairs. Although third generation sequencing techniques (e.g., nanopore sequencing and/or the like) may be capable of resolving longer sequences, the long-read sequences output by applying these third generation sequencing techniques tend to be noisy read sequences containing errors such as insertions, deletions, and substitutions of one or more nucleotide bases. As such, in some cases, noisy read sequences may undergo denoising in order to correct for discrepancies between the noisy read sequences and the original scaffold sequence. In particular, the present disclosure describes various blind denoising techniques in which the noisy read sequences may be denoised in the absence of the original scaffold sequence. That is, in instances where the original scaffold sequence is unavailable, which is often the case, the noisy read sequences may still be denoised by applying the blind denoising techniques described herein.

In some example embodiments, a sequencing controller may perform a blind denoising of multiple read sequences associated with an original scaffold sequence. For example, each read sequence may be a repeat of the original scaffold sequence. Moreover, at least one of the read sequences may be a noisy read sequence (e.g., a noisy repeat) that includes an insertion, a deletion, or a substitution of at least one nucleotide base present in the original scaffold sequence. The sequencing controller may be trained to denoise the read sequences and generate a denoised scaffold sequence without access to and/or knowledge of the original scaffold sequence. That is, the original scaffold sequence may be absent such that the sequence of nucleotide bases in the original scaffold sequence may remain unknown (as in, “blind”) throughout the denoising of the corresponding read sequences. The sequencing controller may be able to generate, based on the least one noisy read sequence (e.g., having the insertion, the deletion, or the substitution of at least one nucleotide base present in the scaffold sequence), the denoised scaffold sequence such that the sequence of nucleotide bases in the denoised scaffold sequence corresponds to the sequence of nucleotide bases in the scaffold sequence.

In some example embodiments, the sequencing controller may apply a sequence denoising model to generate the denoised scaffold sequence. The sequence denoising model may generate the denoised scaffold sequence by at least encoding each read sequence to generate a corresponding embedding in a latent space learned by the sequence denoising model during the training of the sequence denoising model. Furthermore, the sequence denoising model may generate an aggregate embedding corresponding to an aggregate of the embedding associated with each read sequence before decoding the aggregate embedding to generate the denoised scaffold sequence. In this context, the latent space may be a theoretical topological space (e.g., a manifold and/or the like) occupied by the embeddings, each of which being a reduced dimension representation of the corresponding read sequence. For example, the sequence denoising model may learn, during training, a variety of latent features present in the read sequences. These latent features may not be directly observable in each read sequence but capture the relationships that exist between different read sequences. Moreover, whereas the read sequence may exhibit numerous features in the sequence space (e.g., N⁴ for a nucleic acid molecule of length N), each read sequence may exhibit fewer latent features. As such, the embedding of a read sequence is said to be a reduced dimension representation of the read sequence while the latent space occupied by the embeddings of the read sequences may have a lower dimensionality (e.g., fewer quantity of dimensions) than the sequence space populated by the read sequences.

In some example embodiments, the sequence denoising model may include a sequence encoder trained to generate, for each read sequence associated with the original scaffold sequence, a corresponding embedding in the latent space. For example, the latent space, which is learned during the training of the sequence denoising model, may be a topological space (e.g., a manifold and/or the like) occupied by the embeddings, each of which being a reduced dimension representation of the corresponding read sequence. In some cases, the sequence encoder may be implemented with a machine learning architecture capable of correlating different portions of each noisy read sequence. For instance, in some cases, the sequence encoder may be implemented with a deep learning architecture having multiple parallel attention mechanisms, such as a transformer and/or the like. Moreover, in some cases, the sequence encoder may be trained to maximize or increase a similarity between the individual embeddings when each read sequence, for example, by minimizing or reducing an edit distance (e.g., a kernelized maximum mean discrepancy (MMD)) between the embeddings when encoding each of the read sequence.

In some example embodiments, the sequence denoising model may include a sequence decoder configured to decode the aggregate embedding corresponding to the aggregate of the embedding associated with each read sequence. In some cases, the sequence decoder may be implemented as a transformer. Moreover, in some cases, the sequence decoder may be configured to decode the aggregate embedding by at least transforming the aggregate embedding from its representation in the latent space into the denoised scaffold sequence in a sequence space also occupied by the read sequences associated with the original scaffold sequence.

In some example embodiments, the sequence denoising model may include a set encoder configured to generate the aggregate embedding corresponding to the aggregate of the embedding associated with each read sequence. For example, the set encoder may be implemented as a set transformer. In some cases, the aggregate embedding may provide a solution or an optimal solution to a loss function associated with the sequencing denoising model, which captures a loss (or error) present in the output of the sequence denoising model in the sequence space occupied by the various sequences as well as the latent space occupied by the corresponding embeddings. Accordingly, the set encoder may be trained to minimize or reduce a first distance between the aggregate embedding and the embeddings of the read sequences in the latent space when determining the aggregate embedding. Moreover, the set encoder may be trained to minimize or reduce a second edit distance between the denoised scaffold sequence and the read sequences in a sequence space when determining the aggregate embedding.

In some example embodiments, the original scaffold sequence may be associated with a variety of a nucleic acid molecule (e.g., DNA, RNA, and/or the like) or variants or derivatives thereof (e.g., single stranded DNA). Accordingly, the nucleic acid molecule may be analyzed, for example, as a part of a variety of downstream bioinformatics tasks, based at least on the denoised scaffold sequence generated by the sequencing controller to correspond to the original scaffold sequence. For example, in some cases, the scaffold sequence may be associated with at least a portion of a molecule such as an antigen or an antibody. Alternatively, in some cases, the scaffold sequence may be associated with a heavy chain and/or a light chain of a B-cell receptor (BCR), or an alpha chain and/or a beta chain of a T-cell receptor (TCR). Accordingly, in some cases, the denoised scaffold sequence may serve as an identifier (or label) for the protein molecule to which the corresponding nucleic acid sequence is conjugated (e.g., linked through chemically induced covalent bonding between the two molecules). In the aforementioned use case for evaluating the binding non-specificity (or poly-specificity) of antibodies, the sequencing controller may perform blind denoising to denoise the long-read sequences, for example, associated with the nucleic acid sequence label (e.g., oligonucleotide, plasmid, and/or the like) conjugated to an antibody, which may have bound to a target antigen, failed to bind to the target antigen, bound to a non-target antigen, or failed to bind to the non-target antigen. The resulting denoised scaffold sequence may therefore be used to identify the antibody and, in some cases, determine groups of antibodies that bound (or failed to bind) to one or more target and non-target antigens. Another example use case is the blind denoising of the nucleic acid sequence labels conjugated with T cell receptors (TCRs). The resulting denoised scaffold sequence may be used to identify and map the corresponding T cell receptor to the gene that expressed the T cell receptor (e.g., as a surface protein).

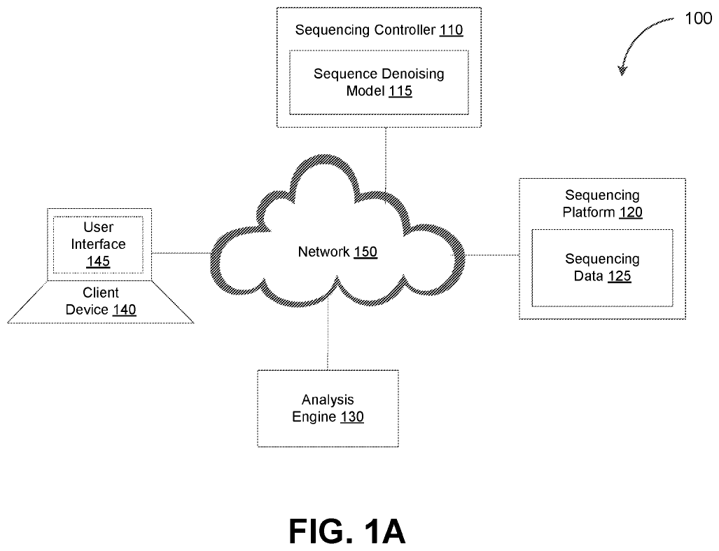

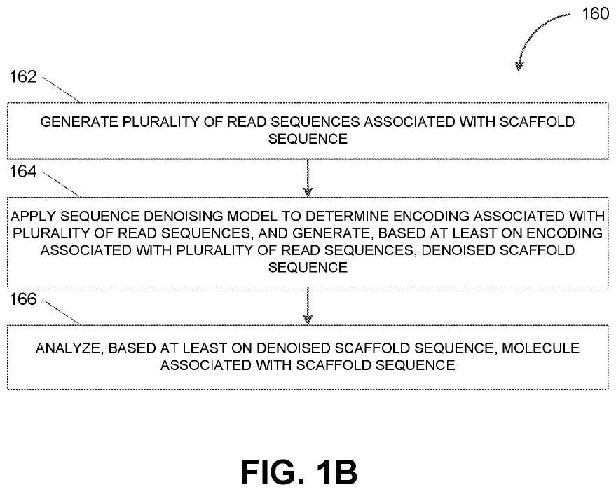

FIG. 1A depicts a system diagram illustrating an example of a sequencing system 100, in accordance with some example embodiments. Referring to FIG. 1A, the sequencing system 100 may include a sequencing controller 110, a sequencing platform 120, an analysis engine 130, and a client 140. As shown in FIG. 1A, the sequencing controller 110, the sequencing platform 120, the analysis engine 130, and the client 140 may be communicatively coupled via a network 150. The client 140 may be a processor-based device including, for example, a workstation, a desktop computer, a laptop computer, a smartphone, a tablet computer, a wearable apparatus, and/or the like. The network 150 may be a wired network and/or a wireless network including, for example, a local area network (LAN), a virtual local area network (VLAN), a wide area network (WAN), a public land mobile network (PLMN), the Internet, and/or the like.

In some example embodiments, the sequencing controller 110 may perform blind denoising of sequencing data, such as sequencing data 125 received from the sequencing platform 125. In some cases, the sequencing data 125 may include multiple noisy read sequences associated with an original scaffold sequence but the denoising of the noisy read sequences may be performed without access to the original scaffold sequence. Moreover, in some cases, the sequencing controller 110 may apply a sequence denoising model 115 to perform the blinding denoising of the sequence data 125. For example, in some cases, the sequence denoising model 115 may include an encoder network fg, a decoder network ge, and a set encoder do. In some cases, the sequence denoising model 115 may undergo self-supervised set learning in order to learn a latent space (or embedding space) M from which sequences can be encoded and decoded as well as interpolated between sets of embeddings. By learning such an embedding space, the sequence denoising model 115 may generate and decode an approximation the true embedding of the original scaffold sequence s. For instance, in some cases, the sequence denoising model 115 may ingest, as a part of the sequencing data 125, a set of noisy reads {r¹, . . . , r^{m}} and generate a corresponding denoised scaffold sequence ŝ. As described in more detail below, each read sequence may be processed by the encoder network f_{φ} to generate a set of embeddings {f_{φ}(r¹), . . . , f_{φ}(r^{m})}. The set of embeddings may then processed by the set encoder a_{φ} to generate a single aggregate embedding for the entire set denoted, in some cases, as a_{φ,φ}(R)=a_{φ}({f_{φ}(r¹), . . . , f_{φ}(r^{m})}). To recover the original scaffold sequence s, the decoder network g_{θ} may decode the aggregate embedding to generate the denoised scaffold sequence ŝ, which approximates the original scaffold sequence s.

To further illustrate, FIG. 1B depicts a flowchart illustrating an example of a process 160 for denoising multiple read sequences associated with a scaffold sequence absent the scaffold sequence, in accordance with some example embodiments. Referring to FIGS. 1A-B, the process 160 may be performed by the sequencing controller 110 to denoise, for example, the noisy read sequences included in the sequencing data 125. The denoising of the noisy read sequences may be blind, meaning that the sequence of nucleotide bases forming the corresponding scaffold sequence remains unknown during the denoising of the noisy read sequences. As described in more detail below, in some cases, the sequence controller 110 may apply the sequence denoising model 115 to denoise the noisy read sequences and generate a denoised scaffold sequence corresponding to the original scaffold sequence.

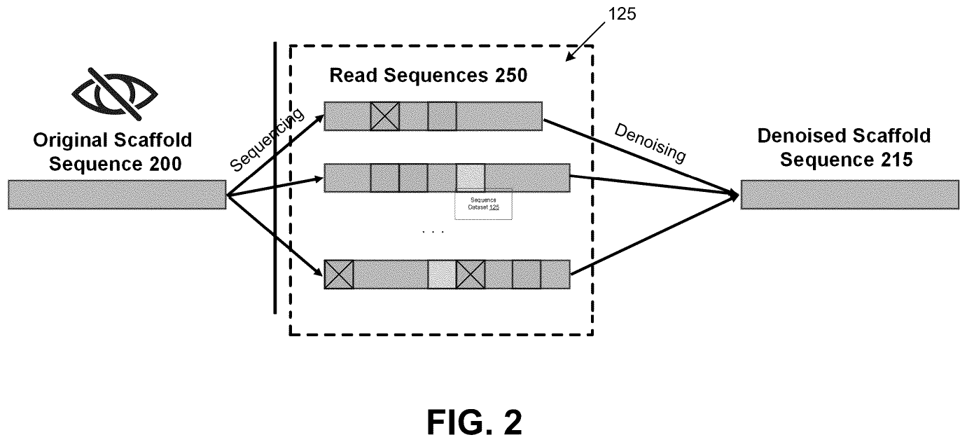

At 162, a plurality of read sequences associated with a scaffold sequence may be received. For example, in some example embodiments, the sequence controller 110 may receive, from the sequencing platform 120, the sequencing data 125. As shown in FIG. 2, the sequencing data 125 may include a plurality of read sequences 250 associated with an original scaffold sequence 200. In some cases, the sequencing platform 120 may apply a variety of sequencing techniques (e.g., next generation sequencing techniques, third generation sequencing techniques, and/or the like) to generate each of the plurality of read sequences 250. Each read sequence of the plurality of read sequences 250 may be a repeat of the original scaffold sequence 200. That is, each read sequence of the plurality of read sequences 250 may constitute a separate inference of the sequence of nucleic acid bases forming the original scaffold sequence 200. Moreover, at least one read sequence of the plurality of read sequences 250 may be a noisy read sequence having an insertion, a deletion, or a substitution of at least one nucleotide base present in the original scaffold sequence 200. Accordingly, at least one read sequence of the plurality of read sequences 250 does not match the original scaffold sequence 200. Without adequate denoising, the plurality of read sequences 250 may not provide a sufficiently accurate reconstruction of the original scaffold sequence 200 for downstream bioinformatics tasks performed at the analysis engine 130 such as, for example, the evaluation of antigen binding non-specificity (or poly-specificity).

At 164, a sequence denoising model may be applied to determine an encoding associated with the plurality of read sequences, and generate, based at least on the encoding associated with the plurality of read sequences, a denoised scaffold sequence. In some example embodiments, the sequencing controller 110 may apply the sequence denoising model 115 to perform a blinding denoising the plurality of read sequences 250. As shown in FIG. 2, the sequencing controller 110 may perform a blind denoising of the plurality of read sequences 250 included in the sequencing data 125 in order to generate a denoised scaffold sequence 215 corresponding to the original scaffold sequence 200. That is, the sequencing controller 110 may denoise the read sequences 250 absent the original scaffold sequence 200 or without access thereto such that the sequence of nucleotide bases in the original scaffold sequence 200 remains unknown throughout the denoising of the plurality of read sequences 250. In some cases, the sequence controller 110 may apply the sequence denoising model 115, which may denoise the plurality of read sequences 250 and generate the denoised scaffold sequence 215 by at least generating an aggregate embedding of the plurality of read sequences 250. For example, in some cases, the sequence denoising model 115 may include a sequence encoder 310 configured to encode each read sequence of the plurality of read sequences 250 and generate a corresponding embedding. Moreover, the sequence denoising model 115 may include a set encoder 330 configured to generate an aggregate embedding corresponding to an aggregate of the embeddings associated with the plurality of read sequences 250 before a sequence decoder 320 of the sequence denoising model 115 decodes the aggregate embedding to generate the denoised scaffold sequence 215. In some cases, the sequence encoder 310, the sequence decoder 320, and the set encoder 330 may be implemented using machine learning models. For instance, in some cases, the sequence encoder 310 may be implemented as a first machine learning model (e.g., a first transformer), the sequence decoder 320 may be implemented as a second machine learning model (e.g., a second transformer), and the set encoder 330 may be implemented as a third machine learning model (e.g., a set transformer).

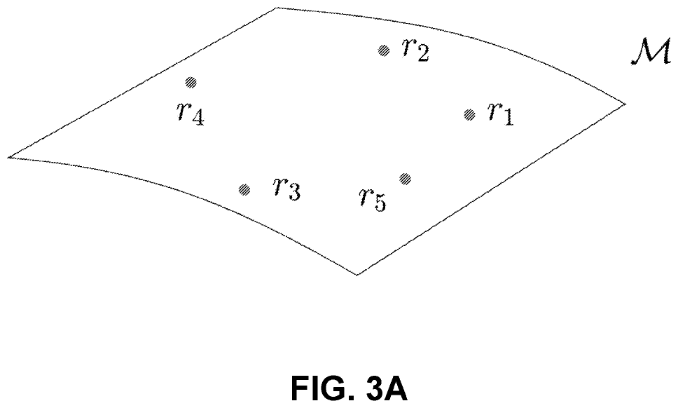

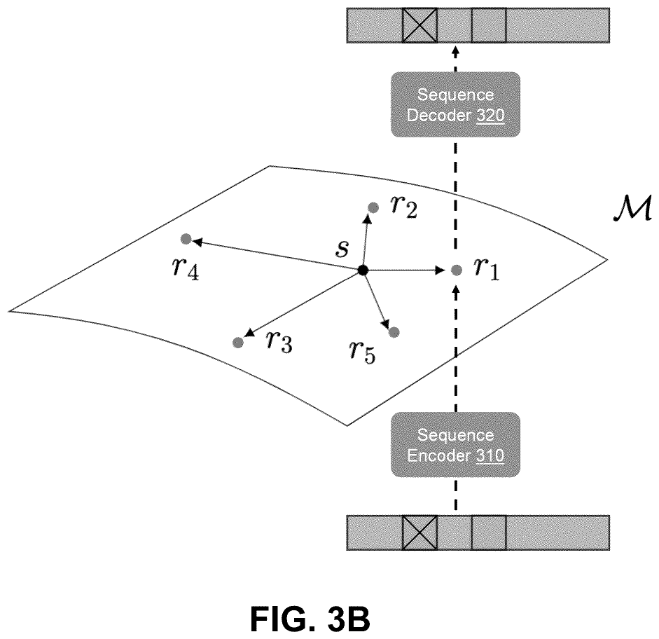

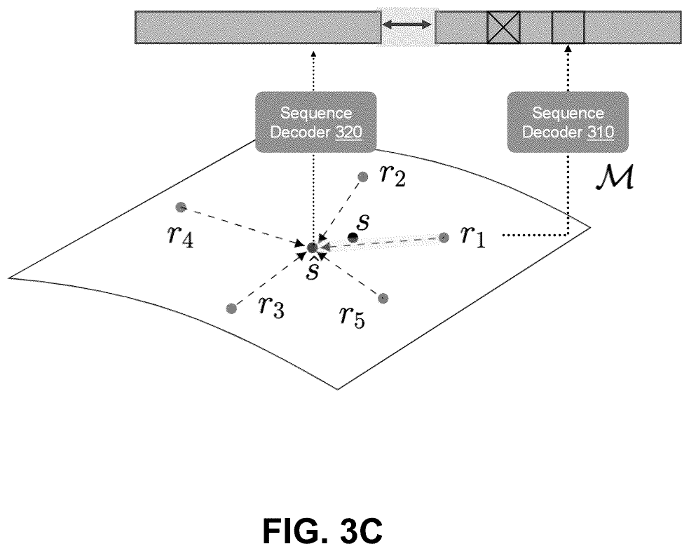

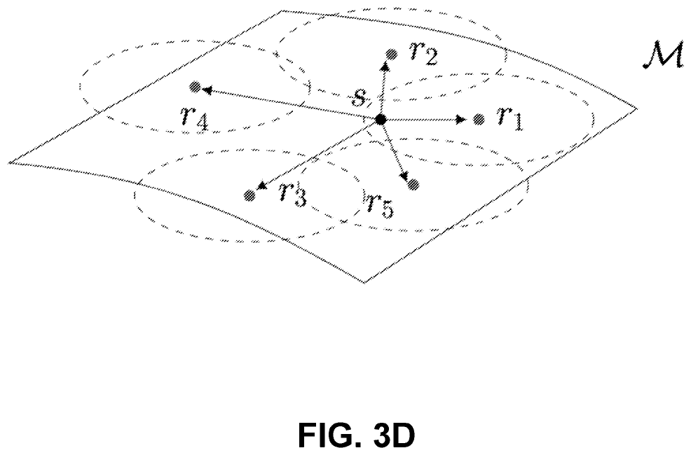

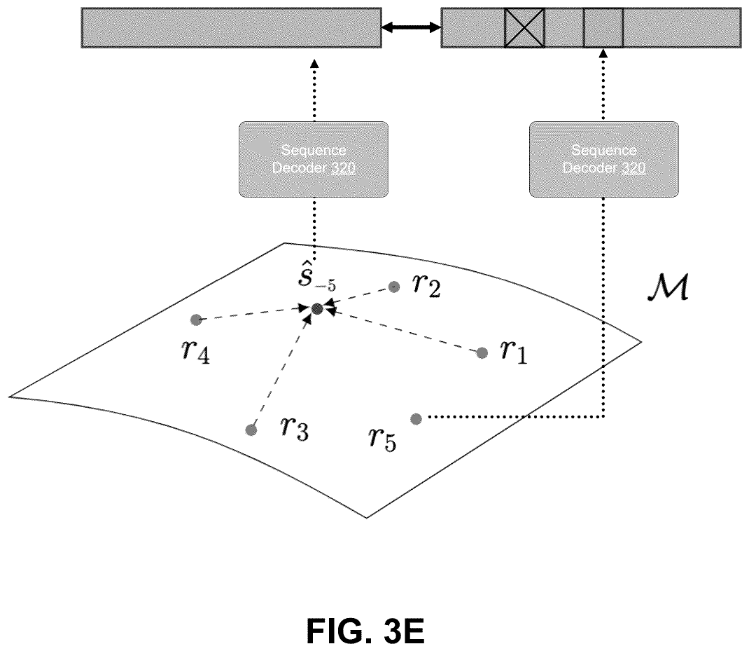

In some example embodiments, the embeddings associated with the plurality of read sequences 250 may occupy a latent space learned by the sequence denoising model 115 during training. Accordingly, the encoding of each read sequence of the plurality of read sequences 250 may include transforming the read sequence from its representation in a sequence space to a corresponding embedding in the latent space. That is, in some cases, the sequence encoder 310 may, for each read sequence of the plurality of read sequences 250, an embedding corresponding to a reduced dimension representation of the read sequence. In this context, the latent space may be a data distribution (e.g., a topological space such as a manifold) occupied by the embeddings of different sequences of nucleotide bases including the embeddings of the plurality of read sequences 250 and, as will be explained in more details below, the aggregate embedding associated with the plurality of read sequences 250. Further illustration is provided at exemplary FIGS. 3A-3C, which show denoising of read sequences.

At 166, a molecule associated with the scaffold sequence may be analyzed based at least on the denoised scaffold sequence. As noted, the original scaffold sequence 200 may be associated with at least a portion of a molecule such as an antigen or an antibody (e.g., a heavy chain and/or a light chain of a B-cell receptor (BCR), an alpha chain and/or a beta chain of a T-cell receptor (TCR), and/or the like). Accordingly, denoising the plurality of read sequences 250 associated with the original scaffold sequence 200 to generate the denoised scaffold sequence 215 while the original scaffold sequence 200 is absent may enable a variety of downstream bioinformatics analyzing the molecule having the original scaffold sequence 200 to be performed based on the denoised scaffold sequence 215. For instance, as one example use case, the analysis engine 130 may evaluate various antibodies for binding non-specificity (or poly-specificity) based on the denoised scaffold sequence of the nucleic acid sequence labels (or tags) conjugated with individual antibodies to differentiate between the antibodies that binds (or fails to bind) to one or more target and/or non-target antigens. Another example use case includes the denoising the nucleic acid sequence labels (or tags) conjugated to a T cell receptor (TCR) such that different T cell receptors may be mapped, based on the corresponding denoised scaffold sequence, to the gene that expressed the T cell receptor (as a surface protein).

Denoising of the plurality of read sequences 250 in the aforementioned manner to generate the denoised scaffold sequence 215 may ensure that the denoised scaffold sequence 215 is a sufficiently accurate reconstruction of the original scaffold sequence 200 for the downstream bioinformatics tasks. For example, in some cases, the original scaffold sequence 200 may be associated with at least a portion of an antigen or an antibody. Alternatively, in some cases, the original scaffold sequence 200 may be associated with a heavy chain and/or a light chain of a B-cell receptor (BCR), or an alpha chain and/or a beta chain of a T-cell receptor (TCR). Accordingly, in some cases, the denoised scaffold sequence 215 may serve as a molecular identifier for the corresponding protein molecule and/or the cell expressing the protein molecule. For instance, in cases where the sequencing data 125 is used to evaluate the binding non-specificity (or poly-specificity) of antibodies, using the denoised scaffold sequence 215 may enable the correct identification of a binding (or non-binding) antibodies whereas using any of the plurality of read sequences 250 may result in the misidentification of the binding (or non-binding) antibodies. In other cases where the sequencing data 125 is used to map T cell receptors (TCRs) to the corresponding genes, the use of the denoised scaffold sequence 215 may enable a correct disambiguation between the genes expressing different T cell receptors whereas the use of the plurality of read sequences 250 may prevent such mapping from being performed with reasonable accuracy.

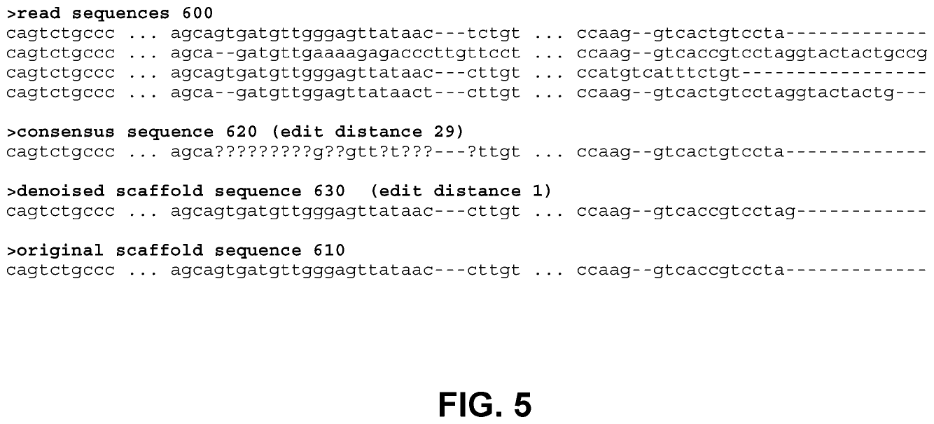

Denoising multiple read sequences, such as the plurality of read sequences 250 in the manner disclosed herein may achieve a better denoising performance than conventional denoising techniques such as multi-sequence alignment (MSA) in which a consensus sequence corresponding to the original scaffold sequence 200 is determined by aligning the plurality of read sequences 250 and identifying the most common nucleotide base at each position. In particular, conventional denoising techniques such as multi-sequence alignment (MSA) may fail to provide adequate denoising performance when there are two few read sequences associated with the original scaffold sequence 200. For example, multi-sequence alignment (MSA) requires at least three read sequences. Moreover, conventional denoising techniques do not respect biological priors (e.g., no stop or frameshift) and are incapable of leveraging global structure within the sequencing data 125. Further illustration is provided at FIG. 5, which depicts an example case where read sequences associated with an antibody light chain are denoised.

FIGS. 3A-3C depict schematic diagrams illustrating the blind denoising of the plurality of read sequences 250 performed by the sequence denoising model 115. For example, FIG. 3A depicts an example of a latent space [symbol] learned by the sequencing controller 110 during training. As noted, in some example embodiments, the sequence encoder 310 may generate, for each read sequence of the plurality of read sequences 250, a corresponding embedding in the latent space [symbol] . In the example shown in FIGS. 3A-3C, the sequence encoder 310 may generate a first embedding r₁ for a first read sequence 250a, a second embedding r₂ for a second read sequence 250b, a third embedding r₃ for a third read sequence 250c, a fourth embedding r₄ for a fourth read sequence 250d, and a fifth embedding r₅ for a fifth read sequence 250e. Moreover, as shown in FIGS. 3A-3C, the set encoder 330 may generate an aggregate embedding ŝ corresponding to an aggregate of the first embedding r₁, the second embedding r₂, the third embedding r₃, the fourth embedding r₄, and the fifth embedding r₅. The decoder 320 of the sequence denoising model 115 may finally generate the denoised scaffold sequence 215 by at least decoding the aggregate embedding ŝ.

In some example embodiments, the aggregate embedding ŝ may provide an optimal solution to the loss function associated with the sequence denoising model 115, which captures a loss (or error) present in the output of the sequence denoising model 115 in the sequence space occupied by the various sequences (e.g., the original scaffold sequence 200, the plurality of read sequences 250, the denoised scaffold sequence 215, and/or the like) as well as in the latent space [symbol] occupied by the corresponding embeddings (e.g., the first embedding r₁, the second embedding r₂, the third embedding r₃, the fourth embedding r₄, and the fifth embedding r₅). Accordingly, the set encoder 330 may be trained to minimize or reduce a first edit distance between the aggregate embedding s and the embeddings r of the read sequences 250 in the latent space M when determining the aggregate embedding s. Moreover, the set encoder 330 may be trained to minimize or reduce a second edit distance between the denoised scaffold sequence 215 decoded from the aggregate embedding s and each of the read sequences 250 in the sequence space when determining the aggregate embedding ŝ. Accordingly, the set encoder 330 may be trained to generate the aggregate embedding s such that the location of the aggregate embedding ŝ in the latent space [symbol] is as close as possible to the location of the embedding s corresponding to the original scaffold sequence 200, although there is no knowledge of the original scaffold sequence 200 or its corresponding embedding s.

In some cases, the loss in the latent space (or embedding space) [symbol] , which includes the edit distance between two or more embeddings of different lengths, may be expressed as d(a_{φ,φ}(R), f_{φ}(rⁱ)). The distance between two or more different length embeddings in the latent space [symbol] may be quantified based on a distance metric d, such as a kernelized maximum mean discrepancy (MMD), computed based on Equation (1) below. The distance d between the aggregate embedding a and the embeddings of individual repeat read sequences f computed based on Equation (1) may correspond to the aforementioned latent space loss function. In Equation (1), if x=a and y=f, then Lₓ may correspond to the length of the aggregate embedding (after length transformation), L_{y} may correspond to the lengths of the repeat read sequences, and k may denote a smoothing kernel.

$$( 1 )$$

$${d}_{k} ( x , y ) = [ \frac{1}{{L}_{x}^{2}} {\sum }_{i , j = 1}^{{L}_{x}} k ( {x}_{i} , {x}_{j} ) - \frac{1}{{L}_{x} {L}_{y}} {\sum }_{i j = 1}^{{L}_{x} {L}_{y}} k ( {x}_{i} , {x}_{j} ) - \frac{1}{{L}_{y}^{2}} {\sum }_{i , j = 1}^{{L}_{y}} k ( {y}_{i} , {y}_{j} ) ]$$

As noted, the decoder 320 of the sequence denoising model 115 may generate the denoised scaffold sequence 215 by at least decoding the aggregate embedding ŝ. In some example embodiments, through regularization of the latent space imposed during the training of the sequence denoising model 115, the decoder 320 may be conditioned to decode between the first embedding r₁, the second embedding r₂, the third embedding r₃, the fourth embedding r₄, and the fifth embedding r₅. Regularization (or conditioning) of the latent space [symbol] may increase its density such that points on the latent space [symbol] not populated by embeddings of individual repeats are still meaningful. For example, without regularization of the latent space [symbol] decoding an embedding occupying a midpoint between two or more repeat read sequences in the latent space [symbol] may not necessarily yield a sensible sequence in the sequence space. Contrastingly, regularization (or conditioning) of the latent space [symbol] increases the likelihood that a sequence decoded from an embedding in the latent space [symbol] yields a sensible sequence in the sequence space.

It should be appreciated that regularization of the latent space may be achieved by applying a variety of regularization techniques. For instance, as shown in FIG. 3D, the regularization of the latent space may be achieved by adding Gaussian noise to each of the first embedding r₁, the second embedding r₂, the third embedding r₃, the fourth embedding r₄, and the fifth embedding r₅, thus distributing the probability mass of each embedding across the latent space. Alternatively and/or additionally, L2 regularization, also known as weight decay or ridge regression, may be applied by introducing a regularization term to the loss function associated with the sequence denoising model 115. Embedding parameters may also undergo regularization in order to force, for example, the first embedding r₁, the second embedding r₂, the third embedding r₃, the fourth embedding r₄, and the fifth embedding r₅ to lie close together to the origin of the latent space. In some cases, the read sequences ingested by the sequence denoising model 115 during training may be masked (e.g., masking one or more base pairs in the read sequences) in order to force the model to use context information. Augmenting the training data in this manner may prevent the sequence denoising model 115 from memorizing the repeat read sequences in the training data. Masking out one or more random base pairs in the read sequences ingested by the sequence denoising model 115 during training may increase the robustness of the sequence denoising model 115. The trained sequence denoising model 115 may be capable of making inferences based on the context base pairs around the masked out base pairs and thus be more tolerant of small omissions in the input read sequences.

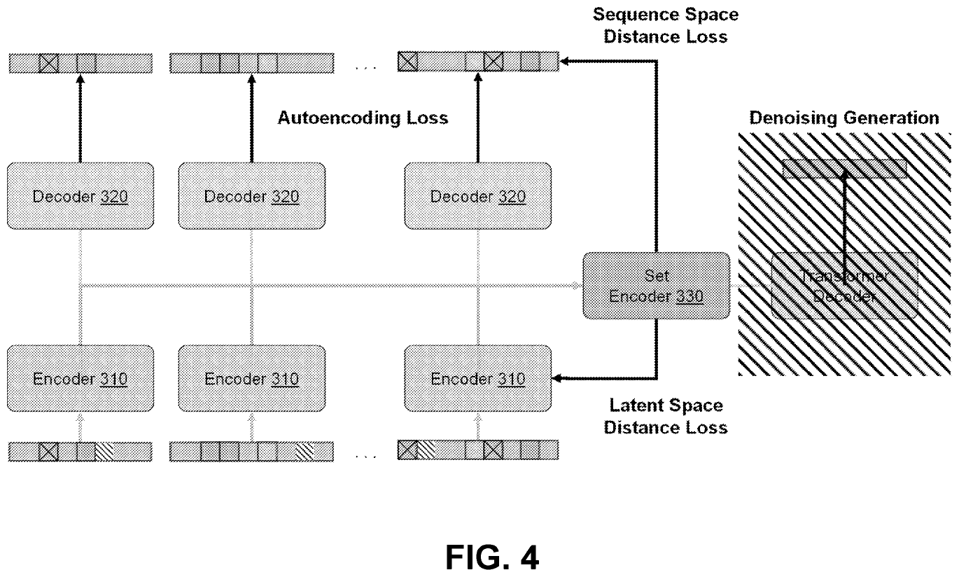

In some example embodiments, the sequence denoising model 115 may be trained in a self-supervised manner to reduce or minimize a loss function of the sequence denoising model 115. In some cases, the loss function of the sequence denoising model 115 may capture an error associated with the sequence encoder 310, the sequence decoder 320, and the set encoder 330. For example, FIG. 4 depicts a schematic diagram illustrating the loss associated with each of the sequence encoder 310, the sequence decoder 320, and the set encoder 330. As shown in FIG. 4, the overall loss function associated with the sequence denoising model 115 may include an autoencoding loss associated with the encoding performed by the encoder 310 and the decoding performed by the decoder 320, a latent space distance loss associated with the aggregate embedding generated by the set encoder 330, and a sequence space distance loss that arise when the aggregate embedding is decoded into its corresponding sequence space representation. The loss function associated with the sequence denoising model 115 may be further expressed as the equation below.

$$arg {min}_{θ , ϕ , Φ} \frac{1}{n} {\sum }_{i = 1}^{n} \frac{1}{m} {\sum }_{j = 1}^{m} {\underbrace{- log {p}_{θ} ( {r}^{j} \vert {f}_{ϕ} ( {r}^{j} ) )}}_{Autoencoding Loss} + {\underbrace{d ( {a}_{Φ} ( \{ {{f}_{Φ} ( {r}^{j} )}_{j = 1}^{m} \} ) , {f}_{ϕ} ( {r}_{j} ) )}}_{Latent Space Distance Loss} - {\underbrace{log {p}_{θ} ( {r}^{j} \vert {a}_{Φ} ( \{ {{f}_{ϕ} ( {r}^{j} )}_{j = 1}^{m} \} ) )}}_{Sequence Space Distance Loss}$$

As noted, the sequence controller 110 may apply the sequence denoising model 115 to denoise the plurality of read sequences 250 while the original scaffold sequence 200 associated with the plurality of read sequences 250 remains unknown. Moreover, in some example embodiments, the sequence denoising model 115 may be trained in a self-supervised manner, for example, by undergoing self-supervised set learning (SSSL), meaning that the sequence denoising model 115 may be trained without any ground truth scaffold sequences.

As such, in some example embodiments, the performance of the sequence denoising model 115 cannot be evaluated based on an edit distance between the denoised scaffold sequences generated by the sequence denoising model 115 and the corresponding ground truth scaffold sequences. Instead, the performance of the sequence denoising model 115 may be evaluated based on a leave-one-out (LOO) edit distance. For example, if the training set used to train the sequence denoising model 115 includes the plurality of read sequences 250, the sequence controller 110 may apply the sequence denoising model 115 to encode all but one of the plurality of read sequences 250. In the example shown in FIG. 3E, the sequence controller 110 may omit the fifth read sequence 250e. The edit distance between the fifth read sequence 250e and the denoised scaffold sequence 215 generated by the sequence denoising model 115 by decoding the aggregate encoding ŝ₋₅ of the first embedding r₁, the second embedding r₂, the third embedding r₃ and the fourth embedding r₄ may serve as a surrogate measure of the performance of the sequence denoising model 115. In some cases, the edit distance between the fifth read sequence 250e and the denoised scaffold sequence 215 generated by the sequence denoising model 115 by decoding the aggregate encoding ŝ₋₅ may correspond to an upper bound for the edit distance between the denoised scaffold sequence 215 and the true original scaffold sequence 210.

FIG. 5 depicts an example use case in which the read sequences 600 associated with a light chain of an antibody are denoised using the sequence denoising model 115 and a conventional multi-sequence alignment (MSA) based denoising technique. The respective performances of each denoising technique may be quantified based on an edit distance relative to an original scaffold sequence 610 of the light chain. For example, as shown in FIG. 5, the consensus sequence 620 generated by applying the conventional multi-sequence alignment (MSA) based technique is associated with an edit distance of 29 whereas the denoised scaffold sequence 630 generated by applying the sequence denoising model 115 is associated with a much smaller edit distance of 1. These results indicate that the sequence denoising model 115 described herein is capable of achieving superior denoising performance when compared to a conventional denoising technique such as multi-sequence alignment (MSA).

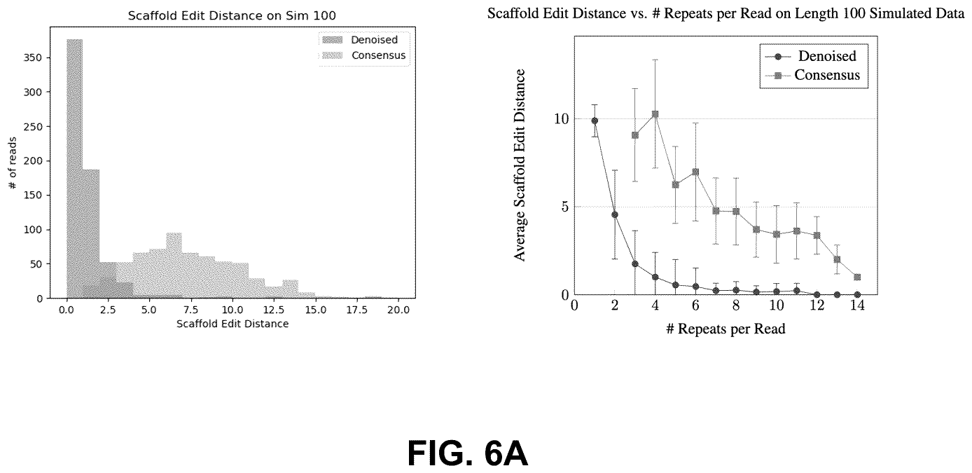

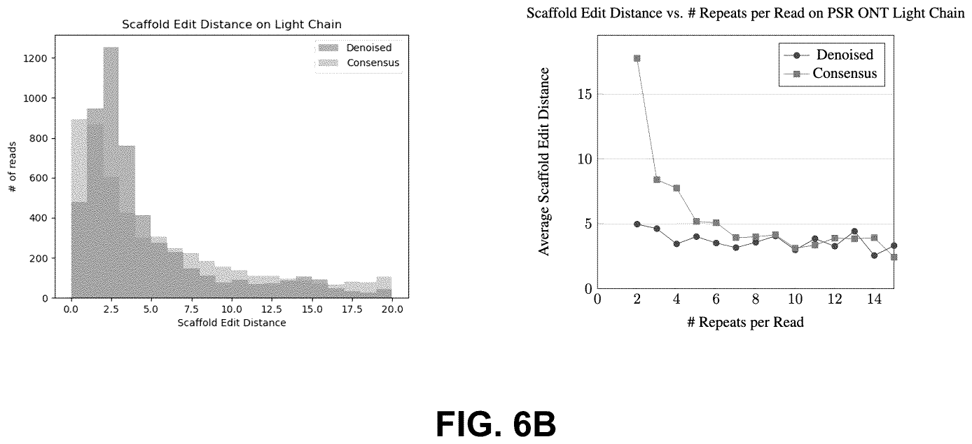

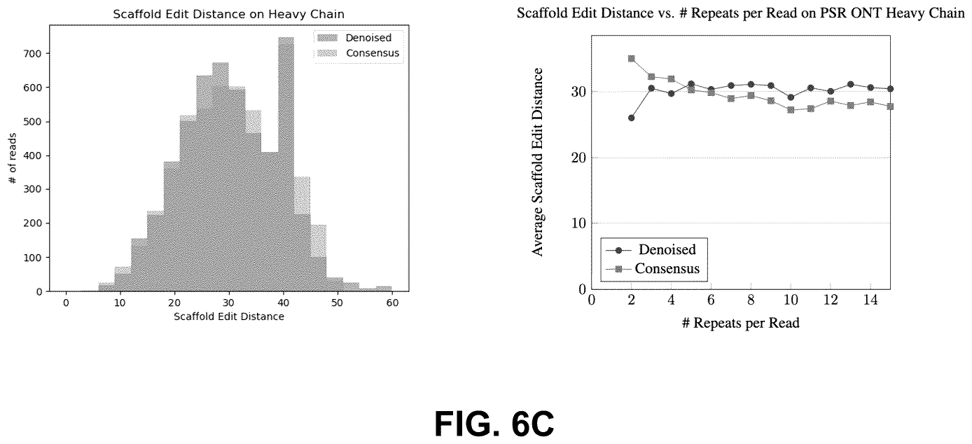

FIGS. 6A-6C depict graphs illustrating a further comparison of the respective performances of the sequence denoising model 115 and the conventional multi-sequence alignment based denoising technique, in accordance with some example embodiments. For example, FIG. 6A depicts graphs illustrating a comparison of the respective performances of the sequence denoising model 115 and the conventional multi-sequence alignment based denoising technique when denoising simulated V/J sequences that are 100 base pairs in length. Repeat sequences may be generated for each simulated V/J sequence to form a total of ten thousand sequences. As shown in FIG. 6A, the sequence denoising model 115 achieved better denoising performance, as quantified by an edit distance relative to the original scaffold sequences, than the conventional multi-sequence alignment based denoising technique. In particular, FIG. 6A shows that the sequence denoising model 115 is capable of achieving a better denoising performance, as indicated by a lower edit distance relative to the original scaffold sequences, even when fewer read sequences are available to reconstruct the original scaffold sequences.

FIG. 6B depicts graphs illustrating a comparison of the respective performances of the sequence denoising model 115 and the conventional multi-sequence alignment based denoising technique when denoising antibody light chains sequenced by Oxford Nanopore Technology sequencing platform. In the example shown in FIG. 6B, one hundred thousand scaffold sequences of an average length of 327 base pairs and an average of eight repeat read sequences per scaffold sequence were denoised using the sequence denoising model 115 and the conventional multi-sequence alignment based denoising technique. FIG. 6B shows that the sequence denoising model 115 outperformed the conventional multi-sequence alignment based denoising technique despite the sequencing data being filtered for success with the conventional multi-sequence alignment based denoising technique. As shown in FIG. 6B, the sequence denoising model 115 outperformed the conventional multi-sequence alignment based denoising technique with, on average across different quantities of available repeat read sequences, a lower edit distance of one base pair.

FIG. 6C depicts graphs illustrating a comparison of the respective performances of the sequence denoising model 115 and the conventional multi-sequence alignment based denoising technique when denoising antibody heavy chains sequenced by. In the example shown in FIG. 6C, one hundred thousand scaffold sequences of an average length of 362 base pairs and an average of eight repeat read sequences per scaffold sequence were denoised using the sequence denoising model 115 and the conventional multi-sequence alignment based denoising technique. Despite the heavy chain of an antibody exhibiting more variations and thus being more challenging to denoise, the sequence denoising model 115 still provided superior performance (e.g., lower edit distance relative to the original scaffold sequences), particularly when more repeat sequences were available per scaffold sequence.

In view of the above-described implementations of subject matter this application discloses the following list of examples, wherein one feature of an example in isolation or more than one feature of said example taken in combination and, optionally, in combination with one or more features of one or more further examples are further examples also falling within the disclosure of this application:

Item 1: A computer-implemented method, comprising: receiving a plurality of read sequences associated with a scaffold sequence, each read sequence of the plurality of read sequences comprising a repeat of the scaffold sequence, and at least one read sequence of the plurality of read sequences being a noisy read sequence that fails to match the scaffold sequence; applying a sequence denoising model trained to determine an encoding associated with the plurality of read sequences, and generate, based at least on the encoding associated with the plurality of read sequences, a denoised scaffold sequence corresponding to the scaffold sequence; and analyzing, based at least on the denoised scaffold sequence, a molecule associated with the scaffold sequence.

Item 2: The method of Item 1, wherein the sequence denoising model generates the denoised scaffold sequence absent the scaffold sequence.

Item 3: The method of any of Items 1 to 2, wherein the sequence denoising model generates the denoised scaffold sequence by at least encoding the plurality of read sequences to generate a plurality of embeddings, determining an aggregate embedding corresponding to an aggregate of the plurality of embeddings, and decoding the aggregate embedding to generate the denoised scaffold sequence.

Item 4: The method of Item 3, wherein the sequence denoising model includes a sequence encoder trained to generate the plurality of embeddings by at least encoding the plurality of read sequences.

Item 5: The method of Item 4, wherein the sequence encoder is trained to increase a similarity between the plurality of embeddings when encoding the plurality of read sequences.

Item 6: The method of Item 5, wherein the sequence encoder increases the similarity between the plurality of embeddings by at least reducing an edit distance between the plurality of embeddings when encoding the plurality of read sequences.

Item 7: The method of Item 6, wherein the edit distance includes a kernelized maximum mean discrepancy (MMD).

Item 8: The method of any of Items 4 to 7, wherein the sequence encoder is trained to generate, for each read sequence of the plurality of read sequences, a corresponding embedding in a latent space.

Item 9: The method of Item 8, wherein the latent space is occupied by a reduced dimension representation of the plurality of read sequences.

Item 10: The method of any of Items 8 to 9, wherein the latent space comprises a topological space occupied by the plurality of embeddings.

Item 11: The method of any of Items 8 to 10, wherein the latent space comprises a manifold.

Item 12: The method of any of Items 4 to 11, wherein the sequence encoder is a transformer.

Item 13: The method of any of Items 3 to 12, wherein the sequence denoising model includes a sequence decoder trained to decode the aggregate embedding to generate the denoised scaffold sequence.

Item 14: The method of Item 13, wherein the decoding of the aggregate embedding includes transforming the aggregate embedding from its latent space representation into the denoised scaffold sequence in a sequence space.

Item 15: The method of any of Items 13 to 14, wherein the sequence decoder includes a transformer.

Item 16: The method of any of Items 3 to 15, wherein the sequence denoising model includes a set encoder trained to determine the aggregate embedding.

Item 17: The method of Item 16, wherein the aggregate embedding determined by the set encoder is invariant to an ordering of the plurality of read sequences.

Item 18: The method of any of Items 16 to 17, wherein the set encoder is trained to reduce a first distance between the aggregate embedding and the plurality of embeddings in a latent space when determining the midpoint embedding.

Item 19: The method of Item 18, wherein the set encoder is further trained to reduce a second distance between the denoised scaffold sequence and the plurality of read sequences in a sequence space when determining the aggregate embedding.

Item 20: The method of any of Items 16 to 19, wherein the set encoder includes a set transformer.

Item 21: The method of any of Items 1 to 20, wherein the noisy read sequence includes at least one insertion, deletion, or substitution of a nucleobase type included in the scaffold sequence.

Item 22: The method of any of Items 1 to 21, wherein the scaffold sequence is a nucleic acid sequence label conjugated with at least a portion of an antigen or an antibody.

Item 23: The method of any of Items 1 to 22, wherein the scaffold sequence is a nucleic acid sequence label conjugated with a heavy chain and/or a light chain of a B-cell receptor.

Item 24: The method of any of Items 1 to 23, wherein the scaffold sequence is a nucleic acid sequence label conjugated with an alpha chain and/or a beta chain of a T-cell receptor.

Item 25: The method of any of Items 1 to 24, wherein the scaffold sequence is a deoxyribonucleic acid (DNA) sequence or a ribonucleic acid (RNA) sequence.

Item 26: The method of any of Items 1 to 25, further comprising: training, based at least on a training set, the sequence denoising model to generate one or more denoised scaffold sequences while one or more corresponding scaffold sequences are unknown; determining a performance of the sequence denoising model while the one or more corresponding scaffold sequences is unknown; and upon determining that the performance of the sequence denoising model satisfies one or more thresholds, applying the sequence denoising model to determine the encoding associated with the plurality of read sequences, and generate, based at least on the encoding associated with the plurality of read sequences, the denoised scaffold sequence corresponding to the scaffold sequence.

Item 27: The method of Item 26, wherein the performance of the sequence denoising model is determined based at least on a leave-one-out edit distance of the sequence denoising model.

Item 28: The method of Item 27, further comprising: determining the leave-one-out edit distance associated with the sequence denoising model by at least determining an edit distance between one read sequence from the training set and a denoised scaffold sequence generated by the sequence denoising model based on one or more remaining read sequences in the training set.

Item 29: A system, comprising: at least one data processor; and at least one memory storing instructions, which when executed by the at least one data processor, result in operations comprising the method of any of Items 1 to 28.

Item 30: A non-transitory computer readable medium storing instructions, which when executed by at least one data processor, result in operations comprising the method of any of Items 1 to 28.

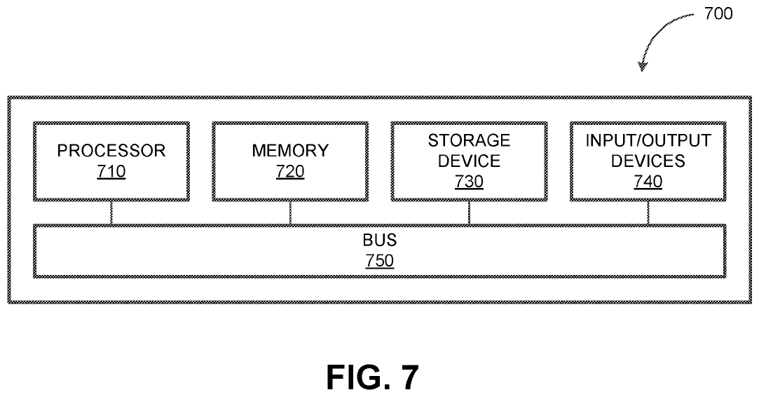

FIG. 7 depicts a block diagram illustrating an example of computing system 700, in accordance with some example embodiments. Referring to FIGS. 1A and 7, the computing system 700 may be used to implement the sequencing controller 110, the sequencing platform 120, the analysis engine 130, the client 140, and/or any components therein.

As shown in FIG. 7, the computing system 700 can include a processor 710, a memory 720, a storage device 830, and an input/output device 740. The processor 710, the memory 720, the storage device 830, and the input/output device 740 can be interconnected via a system bus 750. The processor 710 is capable of processing instructions for execution within the computing system 700. Such executed instructions can implement one or more components of, for example, the EV profile analysis engine 110, the sequencing platform 120, the analysis engine 130, the client 140, and/or the like. In some example embodiments, the processor 710 can be a single-threaded processor. Alternatively, the processor 710 can be a multi-threaded processor. The processor 710 is capable of processing instructions stored in the memory 720 and/or on the storage device 830 to display graphical information for a user interface provided via the input/output device 740.

The memory 720 is a computer readable medium such as volatile or non-volatile that stores information within the computing system 700. The memory 720 can store data structures representing configuration object databases, for example. The storage device 830 is capable of providing persistent storage for the computing system 700. The storage device 830 can be a floppy disk device, a hard disk device, an optical disk device, or a tape device, or other suitable persistent storage means. The input/output device 740 provides input/output operations for the computing system 700. In some example embodiments, the input/output device 740 includes a keyboard and/or pointing device. In various implementations, the input/output device 740 includes a display unit for displaying graphical user interfaces.

According to some example embodiments, the input/output device 740 can provide input/output operations for a network device. For example, the input/output device 740 can include Ethernet ports or other networking ports to communicate with one or more wired and/or wireless networks (e.g., a local area network (LAN), a wide area network (WAN), the Internet).

In some example embodiments, the computing system 700 can be used to execute various interactive computer software applications that can be used for organization, analysis and/or storage of data in various formats. Alternatively, the computing system 700 can be used to execute any type of software applications. These applications can be used to perform various functionalities, e.g., planning functionalities (e.g., generating, managing, editing of spreadsheet documents, word processing documents, and/or any other objects, etc.), computing functionalities, communications functionalities, etc. The applications can include various add-in functionalities or can be standalone computing products and/or functionalities. Upon activation within the applications, the functionalities can be used to generate the user interface provided via the input/output device 740. The user interface can be generated and presented to a user by the computing system 700 (e.g., on a computer screen monitor, etc.).

One or more aspects or features of the subject matter described herein can be realized in digital electronic circuitry, integrated circuitry, specially designed ASICs, field programmable gate arrays (FPGAs) computer hardware, firmware, software, and/or combinations thereof. These various aspects or features can include implementation in one or more computer programs that are executable and/or interpretable on a programmable system including at least one programmable processor, which can be special or general purpose, coupled to receive data and instructions from, and to transmit data and instructions to, a storage system, at least one input device, and at least one output device. The programmable system or computing system may include clients and servers. A client and server are generally remote from each other and typically interact through a communication network. The relationship of client and server arises by virtue of computer programs running on the respective computers and having a client-server relationship to each other.

These computer programs, which can also be referred to as programs, software, software applications, applications, components, or code, include machine instructions for a programmable processor, and can be implemented in a high-level procedural and/or object-oriented programming language, and/or in assembly/machine language. As used herein, the term “machine-readable medium” refers to any computer program product, apparatus and/or device, such as for example magnetic discs, optical disks, memory, and Programmable Logic Devices (PLDs), used to provide machine instructions and/or data to a programmable processor, including a machine-readable medium that receives machine instructions as a machine-readable signal. The term “machine-readable signal” refers to any signal used to provide machine instructions and/or data to a programmable processor. The machine-readable medium can store such machine instructions non-transitory, such as for example as would a non-transient solid-state memory or a magnetic hard drive or any equivalent storage medium. The machine-readable medium can alternatively or additionally store such machine instructions in a transient manner, such as for example, as would a processor cache or other random access memory associated with one or more physical processor cores.

To provide for interaction with a user, one or more aspects or features of the subject matter described herein can be implemented on a computer having a display device, such as for example a cathode ray tube (CRT) or a liquid crystal display (LCD) or a light emitting diode (LED) monitor for displaying information to the user and a keyboard and a pointing device, such as for example a mouse or a trackball, by which the user may provide input to the computer. Other kinds of devices can be used to provide for interaction with a user as well. For example, feedback provided to the user can be any form of sensory feedback, such as for example visual feedback, auditory feedback, or tactile feedback; and input from the user may be received in any form, including acoustic, speech, or tactile input. Other possible input devices include touch screens or other touch-sensitive devices such as single or multi-point resistive or capacitive track pads, voice recognition hardware and software, optical scanners, optical pointers, digital image capture devices and associated interpretation software, and the like.

In the descriptions above and in the claims, phrases such as “at least one of” or “one or more of” may occur followed by a conjunctive list of elements or features. The term “and/or” may also occur in a list of two or more elements or features. Unless otherwise implicitly or explicitly contradicted by the context in which it is used, such a phrase is intended to mean any of the listed elements or features individually or any of the recited elements or features in combination with any of the other recited elements or features. For example, the phrases “at least one of A and B;” “one or more of A and B;” and “A and/or B” are each intended to mean “A alone, B alone, or A and B together.” A similar interpretation is also intended for lists including three or more items. For example, the phrases “at least one of A, B, and C;” “one or more of A, B, and C;” and “A, B, and/or C” are each intended to mean “A alone, B alone, C alone, A and B together, A and C together, B and C together, or A and B and C together.” Use of the term “based on,” above and in the claims is intended to mean, “based at least in part on,” such that an unrecited feature or element is also permissible.

The subject matter described herein can be embodied in systems, apparatus, methods, and/or articles depending on the desired configuration. The implementations set forth in the foregoing description do not represent all implementations consistent with the subject matter described herein. Instead, they are merely some examples consistent with aspects related to the described subject matter. Although a few variations have been described in detail above, other modifications or additions are possible. In particular, further features and/or variations can be provided in addition to those set forth herein. For example, the implementations described above can be directed to various combinations and subcombinations of the disclosed features and/or combinations and subcombinations of several further features disclosed above. In addition, the logic flows depicted in the accompanying figures and/or described herein do not necessarily require the particular order shown, or sequential order, to achieve desirable results. Other implementations may be within the scope of the following claims.

1. A system, comprising:
at least one data processor; and
at least one memory storing instructions, which when executed by the at least one data processor, result in operations comprising:
receiving a plurality of read sequences associated with a scaffold sequence, wherein at least one read sequence of the plurality of read sequences is a noisy read sequence that fails to match the scaffold sequence;
applying a sequence denoising model that
determines an encoding associated with the plurality of read sequences, and
generates, based at least on the encoding associated with the plurality of read sequences, a denoised scaffold sequence corresponding to the scaffold sequence; and
providing, based at least on the denoised scaffold sequence, an analysis of a molecule associated with the scaffold sequence.

2. The system of claim 1, wherein the sequence denoising model generates the denoised scaffold sequence absent the scaffold sequence.

3. The system of claim 1, wherein the sequence denoising model generates the denoised scaffold sequence by at least
encoding the plurality of read sequences to generate a plurality of embeddings,
determining an aggregate embedding corresponding to an aggregate of the plurality of embeddings, and
decoding the aggregate embedding to generate the denoised scaffold sequence.

4. The system of claim 3, wherein the sequence denoising model includes a sequence encoder that generates the plurality of embeddings by at least encoding the plurality of read sequences, and wherein the sequence encoder reduces an edit distance between the plurality of embeddings when encoding the plurality of read sequences.

5. The system of claim 4, wherein the sequence encoder generates, for each read sequence of the plurality of read sequences, a corresponding embedding in a latent space.

6. The system of claim 5, wherein the latent space is a topological space occupied by a reduced dimension representation of the plurality of read sequences.

7. The system of claim 4, wherein the sequence encoder is a transformer.

8. The system of claim 3, wherein the sequence denoising model includes a sequence decoder that generates the denoised scaffold sequence by least decoding the aggregate embedding to generate the denoised scaffold sequence.

9. The system of claim 8, wherein the sequence decoder includes a transformer.

10. The system of claim 3, wherein the sequence denoising model includes a set encoder that determines the aggregate embedding.

11. The system of claim 10, wherein the set encoder reduces a first distance between the aggregate embedding and the plurality of embeddings in a latent space when determining the aggregate embedding, and wherein the set encoder further reduces a second distance between the denoised scaffold sequence and the plurality of read sequences in a sequence space when determining the aggregate embedding.

12. The system of claim 10, wherein the set encoder includes a set transformer.

13. The system of claim 1, wherein the noisy read sequence includes at least one insertion, deletion, or substitution of a nucleobase type included in the scaffold sequence.

14. The system of claim 1, further comprising:
determining, based at least on the denoised scaffold sequence, an identity of the molecule associated with the scaffold sequence.

15. The system of claim 14, further comprising:
determining, based at least on the identity of the molecule conjugated to an antibody, a binding specificity of the antibody.

16. The system of claim 14, further comprising:
identifying, based at least on the identity of the molecule conjugated to a T cell receptor (TCR), a gene expressing the T cell receptor (TCR).

17. The system of claim 1, wherein the scaffold sequence is associated with a nucleic acid sequence label conjugated to (i) a heavy chain or a light chain of a B cell receptor or (ii) an alpha chain and/or a beta chain of a T-cell receptor.

18. The system of claim 1, wherein the scaffold sequence is a deoxyribonucleic acid (DNA) sequence or a ribonucleic acid (RNA) sequence.

19. The system of claim 1, further comprising:
training, based at least on a training set, the sequence denoising model to generate one or more denoised scaffold sequences while one or more corresponding scaffold sequences are unknown;
determining a performance of the sequence denoising model while the one or more corresponding scaffold sequences are unknown; and
upon determining that the performance of the sequence denoising model satisfies one or more thresholds, applying the sequence denoising model to determine the encoding associated with the plurality of read sequences, and generate, based at least on the encoding associated with the plurality of read sequences, the denoised scaffold sequence corresponding to the scaffold sequence.

20. The system of claim 19, further comprising:
determining a leave-one-out edit distance of the sequence denoising model by at least determining an edit distance between one read sequence from the training set and a denoised scaffold sequence generated by the sequence denoising model based on one or more remaining read sequences in the training set; and
determining, based at least on the leave-one-out edit distance, the performance of the sequence denoising model.

21. A computer-implemented method, comprising:
receiving a plurality of read sequences associated with a scaffold sequence, wherein at least one read sequence of the plurality of read sequences is a noisy read sequence that fails to match the scaffold sequence;
applying a sequence denoising model that
determines an encoding associated with the plurality of read sequences, and
generates, based at least on the encoding associated with the plurality of read sequences, a denoised scaffold sequence corresponding to the scaffold sequence; and
providing, based at least on the denoised scaffold sequence, an analysis of a molecule associated with the scaffold sequence.

22. A non-transitory computer readable medium storing instructions, which when executed by at least one data processor, result in operations comprising:
receiving a plurality of read sequences associated with a scaffold sequence, wherein least one read sequence of the plurality of read sequences is a noisy read sequence that fails to match the scaffold sequence;
applying a sequence denoising model that
determines an encoding associated with the plurality of read sequences, and
generates, based at least on the encoding associated with the plurality of read sequences, a denoised scaffold sequence corresponding to the scaffold sequence; and
analyzing, based at least on the denoised scaffold sequence, a molecule associated with the scaffold sequence.

23. The system of claim 1, wherein each read sequence of the plurality of read sequences comprises a separate inference of a sequence of nucleic acid bases forming the scaffold sequence.
